(19) United States
(12) Patent Application Publication
(10) Pub. No.: US 2011/0237993 A1
(43) Pub. Date: Sep. 29, 2011

(54) Posture strap and method of using same

(76) Inventor: Patricia Kirk, Oakland, CA (US)

(21) Appl. No.: 12/798,107

(22) Filed: Mar. 29, 2010

(51) Int. Cl. A61F 5/00

(52) U.S. Cl. 602/20

(57) ABSTRACT

A posture strap formed by connecting the ends of an elongate band to the middle of the band after twisting each end 180 degrees to form two arm loops and the method of correcting posture by disposing the arms of a person into the loops and locating the loops at the upper aspect of the arms while the strap extends along the person's back.

### BACKGROUND

The present invention relates to human posture improvement methods and apparatus and, in particular, to a posture strap that is easily worn either beneath or above clothing.

The relationship of good posture to good health is universally accepted, as is the improvement of health with improved posture. Proper posture broadens the shoulders and opens the chest, which improves lung activity and presents a more attractive appearance.

### BRIEF DESCRIPTION OF THE INVENTION

The present invention is an effective apparatus that, when worn for intermittent periods of time, can re-train the body's musculature to maintain better posture. A flat length of elastic material is configured to form two arm apertures that permit the posture strap of the invention to be disposed on the upper aspect of a person's upper arms and across the back so as to gently draw the shoulders back and thrust the chest forward. The invention causes the shoulders to be positioned more squarely over the hips to better carry the weight of the upper body.

### BRIEF DESCRIPTION OF THE DRAWINGS

FIG. 1 is a perspective view of one embodiment of a posture strap of the present invention;

FIG. 2 is a perspective view of the embodiment of FIG. 1 shown prior to the ends thereof being secured to better illustrate the invention;

FIG. 3 is a perspective view of an embodiment of the invention in which the posture strap is adjustable;

FIG. 4 is a perspective view of a person wearing the posture strap of the invention;

FIG. 5 is a top view of FIG. 4;

FIG. 6 is a perspective view of another embodiment of the invention in which the posture strap is adjustable; and

FIG. 7 is a perspective view of yet another embodiment of the invention in which the posture strap is adjustable.

### DETAILED DESCRIPTION OF THE INVENTION

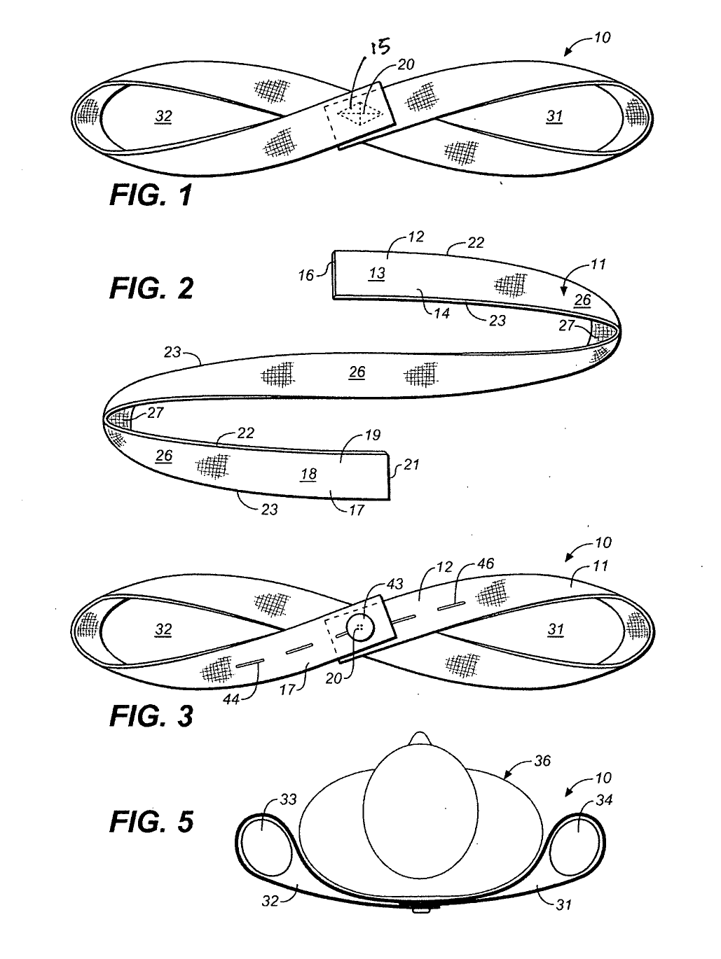

Referring to FIGS. 1 and 2, the posture strap 10 of the present invention comprises a flat elongate band 11 of elastic material having a first end 12 having a first side 13 and an opposing second side 14 and terminating in a first end edge 16. A second end 17 of band 11 has a first side 18 and an opposing second side 19 and terminates in a second end edge 21. A first side edge 22 is between the first end edge 16 and second end edge 21. A second side edge 23 is spaced apart from the first side edge 22 and is between the first end edge 16 and second end edge 21.

A first strap surface 26 is bordered by the first end edge 16 and second end edge 21 and the first side edge 22 and second side edge 23. The first strap surface 26 is contiguous with and includes the first end first side 13 and second end first side 18. A second strap surface 27, opposite the first strap surface 26, is bordered by the first end edge 16 and second end edge 21 and the first and second side edges 22 and 23. The second strap surface 27 is contiguous with and includes the first end second side 14 and the second end second side 19.

The band 11 can advantageously be between 30 and 40 inches in length (between end edges 16 and 21 as measured along the surface 26) and 1 to 3 inches wide (as measured between side edges 22 and 23), although other dimensions could be advantageous in the case of persons of exceptional size (large or small) or depending on the particular elastic material used for the band 11.

The second side 14 of the first end 12 of flat band 11 is attached (as by stitching 15 or similar affixing means) to the first strap surface 26 between the first and second ends 12 and 17 at connection point 20. Connection point 20 is advantageously located at or near the mid-point between first and second ends 12 and 17 as measured along the surface 26. To accomplish this attachment configuration, it is necessary to rotate the band 11 180 degrees between end 17 and connection point 20 prior to attachment. It is important to limit such rotation to just 180 degrees and no more.

The second side 19 of the second end 17 of flat band 11 is attached to the first strap surface 26 between the first and second ends 12 and 17 at connection point 20. To accomplish this, it is necessary to rotate the band 11 180 degrees between end 17 and connection point 20. It is important to limit such rotation to just 180 degrees and no more. The strap ends 12 and 17 overlap at connection point 20 such that one will be in direct contact with first strap surface 26 and the other will be connected to strap surface 26, but in direct contact with the other strap end. As illustrated, the second side 14 of first end 12 is in direct contact with strap surface 26, while the second side 19 of second end 17 overlaps the first surface 13 of first end 12 and is attached to surface 26 there through. As used herein, “attached” or “attachment” means secured thereto without necessitating direct contact.

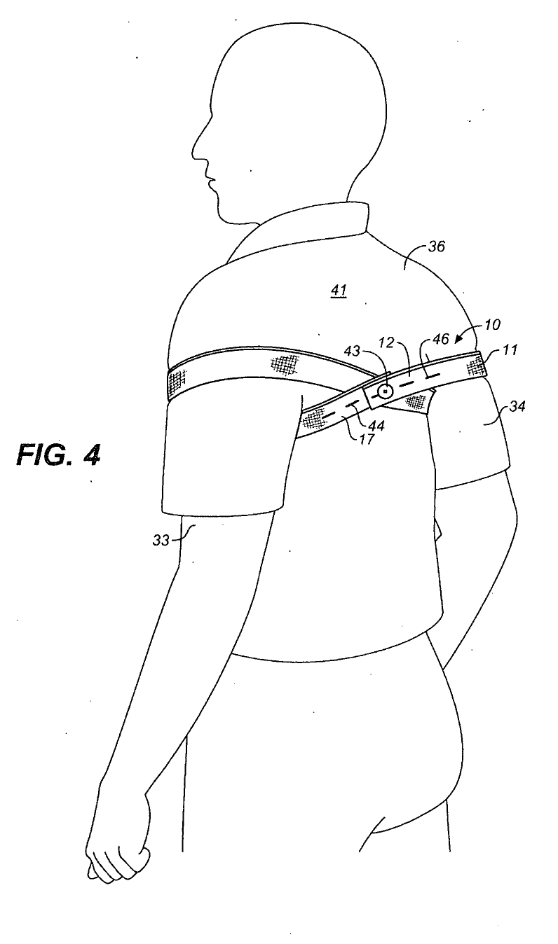

Referring also to FIGS. 4 and 5, the flat band 11 configured as specified above forms two arm loops 31 and 32 adapted to receive the arms 33 and 34 of a person 36. The loops 31 and 32 are best positioned at the upper aspect of the arms 38 and 39 (above the elbow and below the shoulder but closer to the shoulder), with the band 11 extending along the person's back 41. So positioned, the posture strap 10 can be worn either outside of or underneath clothing.

An important feature of the invention that follows from the unique configuration of the band 11 as described above is that when worn properly, all the band surfaces 26 and 27 lay flat against the arms 33 and 34 and back 41 of the person 36 with no twists to cause uncomfortable pressure.

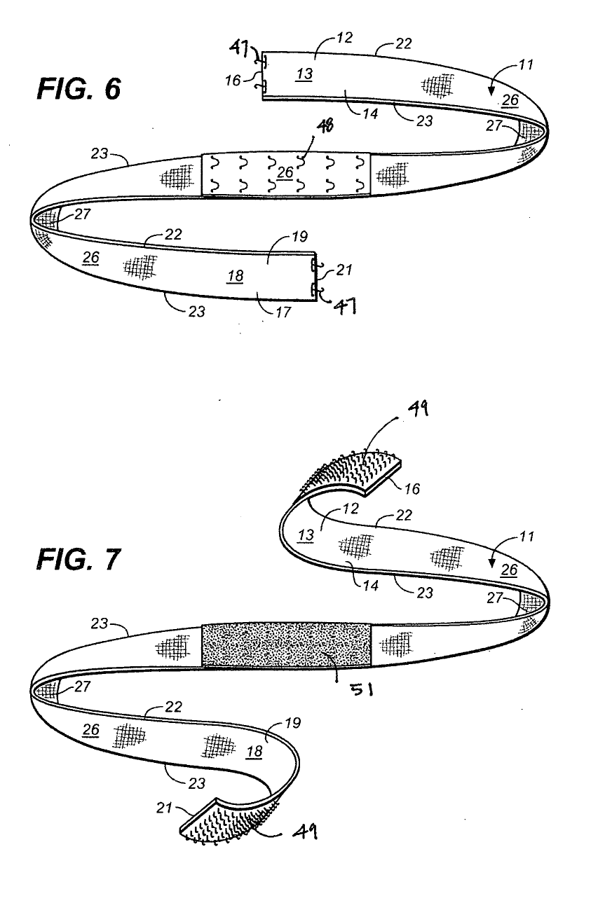

Referring to FIGS. 3, 6 and 7, the first and second ends 12 and 17 of posture strap 10 are attached at connection point 20 by a button 43 and button holes 44 in strap end 17 and button holes 46 in strap end 12. By selecting one of button holes 44 and one of button holes 46 to attach to button 43, the size of the posture strap 10 can be adjusted to different size users and also can be used to vary the amount of force applied. It will be obvious to those skilled in the art that alternatives exist for the button and button hole attachment mechanism, such as hook 47 and eye 48 mechanisms (FIG. 6) or hook 49 and loop 51 materials (i.e. Velcro®), etc. Since the posture strap is put on after the loops are formed and secured, a variable size attachment mechanism need not be limited to those devices that can be manipulated while the posture strap 10 is mounted.

Of course, various changes, modifications and alterations in the teachings of the present invention may be contemplated by those skilled in the art without departing from the intended spirit and scope thereof. As such, it is intended that the present invention only be limited by the terms of the appended claims.

What is claimed is:

1. A posture strap adapted to be attached to the upper arms of a person comprising;
a flat elongate elastic band having
a first end having opposing first and second sides and terminating in a first end edge;
a second end having opposing first and second sides terminating in a second end edge;
a first side edge between said first and second end edges including said first and second ends;
a second side edge spaced apart from said first side edge between said first and second end edges including said first and second ends;
a first side bordered by said first and second end edges and said first and second side edges including said first and second ends wherein said first side of said first end is contiguous with said first side;
a second side opposite said first side and bordered by said first and second end edges and said first and second side edges including said first and second ends wherein said second side of said first end is contiguous with said second side;
said second side of said first end attached to said first side of said elastic band between said first and second ends as measured along said first surface;
said second side of said second end attached to said first side of said elastic band between said first and second ends as measured along said first surface;
wherein loops formed by attaching said second side of said first end and the second side of said second end to said second side of said elastic band are large enough to accommodate the upper arm of a person.

2. The posture strap of claim 1
wherein said second side of said first end of said elastic band is attached to said first side of said elastic band by rotating said band 180 degrees prior to attachment, and
wherein said second side of said second end of said elastic band is attached to said first side of said elastic band by rotating said band 180 degrees prior to attachment.

3. The strap of claim 1 wherein said first and second ends are attached to said second side of said elastic band at the same location on said band.

4. The strap of claim 2 wherein the location where said first and second ends are attached to said second side of said elastic band is approximately mid-way between said first and second end edges as measured along said first side of said band.

5. The strap of claim 1 further comprising:
an attachment mechanism secured to said band at the approximate mid-point between said first and second end edges as measured along said first side of said band; and
a plurality of complementary attachment mechanisms at spaced apart locations along each end of said band whereby each said end can be attached to the approximate mid-point of said band at different locations along said end.

6. The strap of claim 5 wherein said attachment mechanism is a button and said complementary attachment mechanisms are button holes.

7. The strap of claim 5 wherein said attachment mechanism is a plurality of hook eyes and said complementary attachment mechanism is a hook.

8. The strap of claim 5 wherein said attachment mechanism is a length of loops and said complementary attachment mechanism is a length of hooks.

10. A method of improving posture in a person having upper arms between the shoulders and elbows and a back comprising:
forming a posture strap from a flat elongate elastic band having a first end having opposing first and second sides and terminating in a first end edge, a second end having opposing first and second sides terminating in a second end edge, a first side edge between said first and second end edges including said first and second ends, a second side edge spaced apart from said first side edge between said first and second end edges including said first and second ends, a first side bordered by said first and second end edges and said first and second side edges including said first and second ends wherein said first side of said first end is contiguous with said first side, a second side opposite said first side and bordered by said first and second end edges and said first and second side edges including said first and second ends wherein said second side of said first end is contiguous with said second side by attaching said second side of said first end to said first side of said elastic band between said first and second ends as measured along said first surface, attaching said second side of said second end to said first side of said elastic band between said first and second ends as measured along said first surface forming loops large enough to accommodate the upper arms of a person wherein the elongate band provides a connection between the loops; and
disposing the loops of the posture strap at the upper arms of the person with the connection between the loops disposed along the back of the person.

11. The method of claim 10 wherein there are no twists in the elongate band where it contacts the upper arms and the back.
